(19) United States
(12) Patent Application Publication
(10) Pub. No.: US 2015/0305880 A1
(43) Pub. Date: Oct. 29, 2015

(54) PERCUTANEOUS INTERVERTEBRAL ARTIFICIAL DISC REPLACEMENT FOR MINIMAL INVASIVE SURGERY

(71) Applicants: Hyeun Sung Kim, Daejeon Metropolitan City (KR); Hong Won Yoon, Yongin-si (KR)

(72) Inventors: Hyeun Sung Kim, Daejeon Metropolitan City (KR); Hong Won Yoon, Yongin-si (KR)

(21) Appl. No.: 14/569,764

(22) Filed: Dec. 14, 2014

(30) Foreign Application Priority Data
Apr. 24, 2014 (KR) 2014-0049639

(51) Int. Cl. A61F 2/44; A61F 2/46

(52) U.S. Cl. CPC A61F 2/442; A61F 2/4611

(57) ABSTRACT

An artificial disc replacement to be inserted between adjacent vertebrae includes a U shaped disc body to support an upper and a lower vertebrae and to provide elasticity when inserted between adjacent vertebrae; a disc holder located inside of the disc body having protrusions engaging with upper and lower vertebrae through openings at the top and bottom of disc body and to prevent dislocation of the disc from adjacent vertebrae, wherein the protrusions engage with an upper and a lower vertebrae through openings at the top and bottom of artificial disc body; and two bumps each having a coupling blaze facing the upper or lower vertebrae, wherein the disc holder is insertable inside of the disc body and wherein each coupling blaze is engaged by rotating the disc holder.

This application claims priority to Korean Application Serial Application#2014-0049639 filed on 2014 Apr. 24, which issued as Korean Patent 10-1444638 on Sep. 18, 2014, the content of which is incorporated by reference.

### BACKGROUND

The current invention is related to percutaneous intervertebral artificial disc replacement for minimal invasive surgery. More specifically, it's about an intervertebral artificial disc replacement, which can be inserted from patient's back side.

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine as discussed in U.S. Pat. No. 6,419,704. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction). The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter. There are discs between adjacent vertebrae. The outside surfaces of discs are protected with tough fibers. There is the nucleus pulpous inside of discs.

Invertebrate discs have functions of the joint and a crucial role, which minimizes the impact applied to the spine with changing location and shape of nucleus pulposus according to the movement of spine.

The nucleus pulposus is largely composed of water and loses its buffering function since the amount of water decreases with age. Thereby, excessive pressure applied to the fiber causes low back pain. When the degeneration proceeds more, the fibers can be pulled or ruptured. These damaged fibers cause pain at pelvis, legs, by pressing the root of spinal nerve, which is located in behind of the fibers. Hereafter, gradually the space between the vertebral bones will become narrower and side effects such as deformation of the spine may be caused. So, in case of surgery for removing severely distorted or cracked discs, it is necessary to take an action to maintain the space and to prevent the deformation or shaking of spine.

Conventionally, spinal fusion surgery to fix the vertebral bones keeping the normal space using titanium screw was a common procedure. But this conventional procedure caused side effects including low back pain due to fixed vertebrae. Other conventional artificial discs typically have upper and lower plates and an intermediate connector, which is inserted between upper and lower plate. The intermediate connector has a convex shape of rotating plane and upper plate rotates with contacting to the rotating plane. The current artificial discs may cause debris generation in the body of patients because of long-term period friction between upper plate movement and intermediate connector according to the movement of spine. Further, it is expensive to manufacture the upper plate and intermediate connector with special materials for preventing debris generation.

### SUMMARY

In one aspect, an artificial disc replacement to be inserted between adjacent vertebrae includes a U shaped disc body to support an upper and a lower vertebrae and to provide elasticity when inserted between adjacent vertebrae; a disc holder located inside of the disc body having protrusions engaging with upper and lower vertebrae through openings at the top and bottom of disc body and to prevent dislocation of the disc from adjacent vertebrae, wherein the protrusions engage with an upper and a lower vertebrae through openings at the top and bottom of artificial disc body; and two bumps each having a coupling blaze facing the upper or lower vertebrae, wherein the disc holder is insertable inside of the disc body and wherein each coupling blaze is engaged by rotating the disc holder.

In another aspect, a percutaneous intervertebral artificial disc replacement for minimal invasive surgery includes an artificial disc body having a top and bottom supporting an upper and a lower vertebrae, wherein the artificial disc body provides elasticity when inserted between adjacent vertebrae; and an artificial disc holding means positioned inside the artificial disc body with parts engaging the upper and lower vertebrae through openings at the top and bottom of artificial disc body to prevent dislocation of the artificial disc from adjacent vertebrae, wherein the artificial disc holding means has two bumps each having a coupling blaze to contact the upper or lower vertebrae, wherein each coupling blaze is rotatable in the artificial disc holding means.

In yet another aspect, a percutaneous intervertebral artificial disc replacement system for minimal invasive surgery includes an artificial disc replacement inserting device; and an artificial disc body to be positioned by the inserting device between an upper and a lower vertebrae, including: openings at a top and a bottom of the artificial disc body, the artificial disc body supporting the upper and a lower vertebrae; an artificial disc holder positioned inside the artificial disc body and engaging the upper and lower vertebrae through said openings to prevent dislocation of the artificial disc from adjacent vertebrae; and two bumps each having a coupling blaze facing the upper or lower vertebrae, wherein the artificial disc holder is insertable inside of the artificial disc body and each coupling blaze is engaged by rotating the artificial disc holder.

In a further aspect, an artificial disc replacement to be inserted between adjacent vertebrae includes a U shaped disc body to support an upper and a lower vertebrae and to provide elasticity when inserted between adjacent vertebrae; a disc holder located inside of the disc body having protrusions engaging with upper and lower vertebrae through openings at the top and bottom of disc body and to prevent dislocation of the disc from adjacent vertebrae, wherein the protrusions engage with an upper and a lower vertebrae through openings at the top and bottom of artificial disc body; and two bumps each having a coupling blaze facing the upper or lower vertebrae, wherein the disc holder is insertable inside of the disc body and wherein each coupling blaze is engaged by rotating the disc holder.

Implementations of the above aspects for a percutaneous intervertebral artificial disc replacement for minimal invasive surgery may include one or more of the following. The holder is combined into the inside of artificial disc body and prominent parts engage with upper and lower vertebrae through openings at the top and bottom of artificial disc body thereby preventing dislocation of artificial disc from adjacent vertebrae. The prominent parts can be bulges, projections, protrusions or suitable parts that protrude from a surface or thrusts outwardly from a surface. An accepting portion which accommodates the holding means of the artificial disc is formed in the direction of the artificial disc body length inside of the above artificial disc body. The bumps are formed to prevent separation of the artificial disc holding means accommodated. The artificial disc holder can be detachably coupled to the above accepting portion. Further, the artificial disc body includes the first and second upper plate, lower plate and elastic intermediate connector. The upper plate which is divided into two plates, first and second upper plate, has an opening and engages with upper vertebrae. The lower plate has an opening and engages with lower vertebrae. The elastic intermediate connector which connects the first and second upper plate to the lower plate provides elastic force in an opposite direction of load in case the front portion of the first and second upper plate come down due to load of upper vertebrae. The elastic intermediate connector has a vertical groove in its middle side to be bent separately by weight force which has loaded to the each upper plate. Furthermore, the above elastic intermediate connector has horizontal grooves to be easily bent by weight force which loaded to left or right side of top of artificial disc body. The above artificial disc holder or holding means has two bumps which have each coupling blaze toward upper or lower vertebrae. The artificial disc holding means is inserted inside of the artificial disc body and each coupling blaze can be prominent by rotating of the artificial disc holding means in fixed angle.

In other implementations, the above artificial disc holder or holding means may include one or more of the following: a left body coupling portion is coupled with left inside of the above artificial disc body; a bolt through hole has performed in the horizontal direction in the middle of the left coupling portion and there are each coupling hole at left and right side of bolt through hole; the right coupling portion is coupled with right inside of the above artificial disc body; a bolt through hole has performed in the horizontal direction in the middle of the right coupling portion and there are each coupling holes at left and right side of bolt through holes facing opposite of coupling holes of the left coupling portion the upper bump is located between left body coupling portion and right body coupling portion. It rotatably coupled with right coupling hole of left body coupling portion and right body coupling portion; a coupling blaze engages with upper vertebrae by being prominent through the opening performed at upper portion of artificial disc body; the lower bump is located between left body coupling portion and right body coupling portion. It rotatably coupled with left coupling hole of left body coupling portion and right body coupling portion; a coupling blaze engages with lower vertebrae by being prominent through the opening performed at lower portion of artificial disc body;

a bolt portion combines the left body coupling portion and the right body coupling portion by piercing bolt through hole of the left body coupling portion and the right body coupling portion on condition that the upper and lower bumps are coupled with the left body coupling portion on left and the right body coupling portion on right of them; and a nut portion is coupled with a part of the bolt portion which is exposed to outside by piercing the right body coupling portion.

Further, the upper bump may include one or more of the following: a rotatable rod portion whose both end are engaged with the coupling holes of the left body coupling portion and the right body coupling portion; and another rod portion attached to middle of the above rod portion includes a coupling blaze which is prominent through an opening performed at the top of artificial disc, when the left and right body coupling portions were rotated by certain angle. The lower bump can have: a rotatable rod portion whose both ends are engaged with the coupling holes of the left body coupling portion and the right body coupling portion; and a coupling blaze which is prominent through an opening performed at the bottom of artificial disc, when the left and right body coupling portions were rotated by certain angle.

The system can also include a groove matching with the coupling portion of the inserting device is performed on the side of left body coupling portion to rotate holding means of artificial disc by rotating the coupling portion of the inserting device when the artificial disc engaged with the inserting device.

Advantages of the above aspects may include one or more of the following. The system provides a superior percutaneous intervertebral artificial disc replacement for minimal invasive surgery. The system handles the wear problem caused by frequent friction between the upper and lower plate by providing elastic force through inserting the intervertebral artificial disc. The artificial disc provides a disc holder that engages with upper and lower vertebrae through openings at the top and bottom of artificial disc body thus preventing dislocation of artificial disc from adjacent vertebrae. The system provides a minimal invasive artificial disc, which can be inserted through the back of patients by minimal invasive surgery with inserting device for an artificial disc. Additionally, U-shaped artificial disc body has an effect of preventing dislocation of artificial disc replacement from adjacent vertebrae. The artificial disc replacement procedure doesn't need to fix vertebrae with screws and has no side effects such as back pain due to conventional spinal fusion surgery since the artificial disk is similar to human disk in terms of the stability, flexibility and mobility of spinal segment after surgery.

### BRIEF DESCRIPTION OF THE DRAWINGS

FIG. 1 is an isomeric view of a percutaneous intervertebral artificial disc replacement for minimal invasive surgery.

FIG. 2 is a left lateral view of a percutaneous intervertebral artificial disc replacement for minimal invasive surgery.

FIG. 3a is a left lateral view of a percutaneous intervertebral artificial disc replacement for minimal invasive surgery whose bumps are prominent through the top and bottom

FIG. 3b is a posterior view of a percutaneous intervertebral artificial disc replacement for minimal invasive surgery.

FIG. 3c is an anterior view of a percutaneous intervertebral artificial disc replacement for minimal invasive surgery.

FIG. 3d is a superior view of a percutaneous intervertebral artificial disc replacement for minimal invasive surgery.

FIG. 3e is a inferior view of a percutaneous intervertebral artificial disc replacement for minimal invasive surgery.

FIG. 4a is an isomeric view of an artificial disc body.

FIG. 4b is a left lateral view of an artificial disc body.

FIG. 4c is a posterior view of an artificial disc body.

FIG. 4d is a superior view of an artificial disc body.

FIG. 5a is an exploded isomeric view of an artificial disc holding means.

FIG. 5b is an isomeric view of an artificial disc holding means.

FIG. 5c is an isomeric view of an artificial disc holding means which rotated by 90° showing a bump.

FIG. 5d is a left lateral view of an artificial disc holding means.

FIG. 5e is a left lateral view of an artificial disc holding means which rotated by 90°, showing the bump through an opening.

FIG. 6 is an exemplary view of a percutaneous intervertebral artificial disc replacement for minimal invasive surgery engaged with an inserting device.

FIG. 7 is a partial magnified view of an inserting device.

### DETAILED DESCRIPTION

In the following, an embodiment is described in detail according to the present invention with reference to the accompanying drawings.

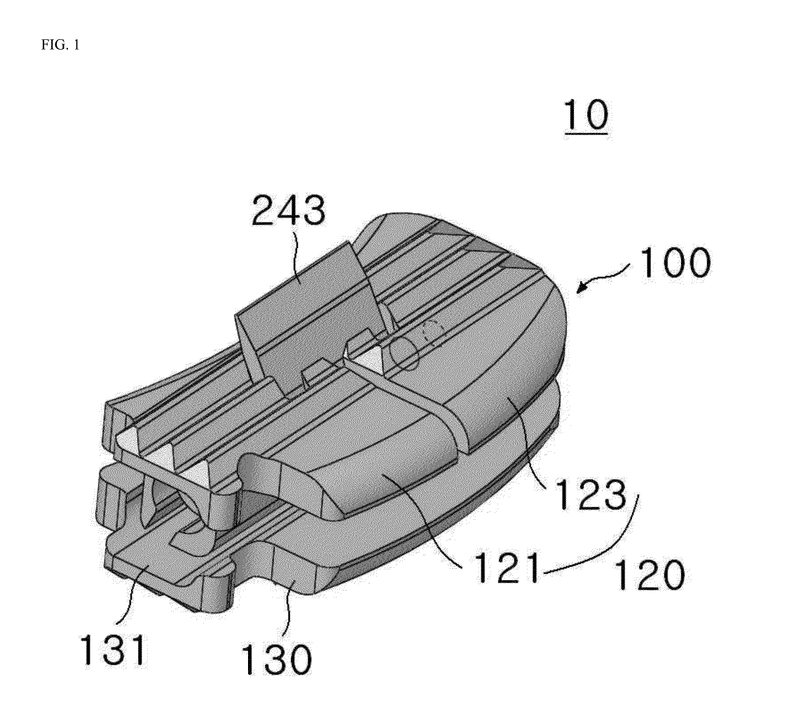

FIG. 1-FIG. 5e show a percutaneous intervertebral artificial disc replacement for minimal invasive surgery 10. The unit includes a U shaped artificial disc body 100 and a holding means (holder) 200 which is inserted between adjacent vertebrae to replace the disc, in case that the disc which compose the lumbar (the hucklebone) has been damaged.

Turning now to FIG. 1, an isomeric view of a percutaneous intervertebral artificial disc replacement for minimal invasive surgery is shown. The artificial disc body 100 has a shape of U character and supports upper and lower vertebrae and provides elasticity by being inserted between adjacent vertebrae. The artificial disc body supports upper and lower vertebrae and provides elasticity by being inserted between adjacent vertebrae.

The holding means 200 is combined into the inside of artificial disc body 100 and prominent bumps 230, 240 engage with upper and lower vertebrae through openings 125 at the top and bottom of artificial disc body thereby it prevents dislocation of artificial disc 10 from adjacent vertebrae. The term prominent relates to parts that project, protrude from a surface, thrust outward or bulging.

The artificial disc holding means 200 has two bumps 240, 230 which have each coupling blaze 243, 233 toward upper or lower vertebrae. The artificial disc holding means is inserted inside of the artificial disc 100 and each coupling blaze 243, 233 can be prominent by rotating of the artificial disc holding means 200 in fixed angle.

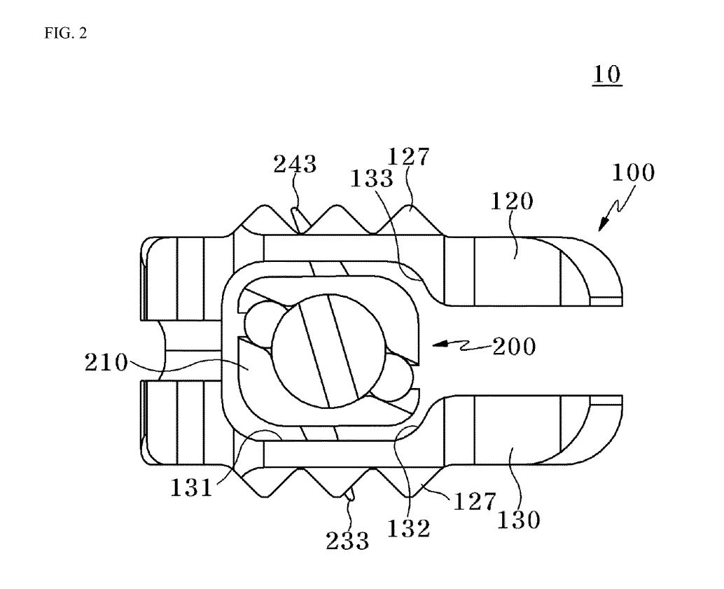

FIG. 2 illustrates an artificial disc holding means 200 combined to the inner accepting portion 131 of the artificial disc body 100. FIG. 2 is a left lateral view of a percutaneous intervertebral artificial disc replacement for minimal invasive surgery. The coupling blazes 243 slightly projecting out through the opening 125 performed at the upper plate 120 is shown. The height of the prominent coupling blaze is similar with the holding bump 127, not severely high. It's same to the prominent coupling blaze 233 projecting out through the opening 125 performed at the lower plate 130.

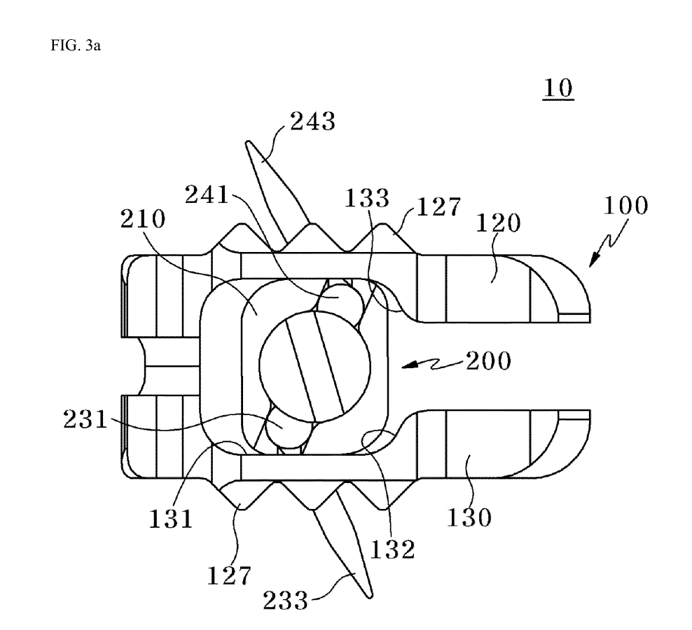

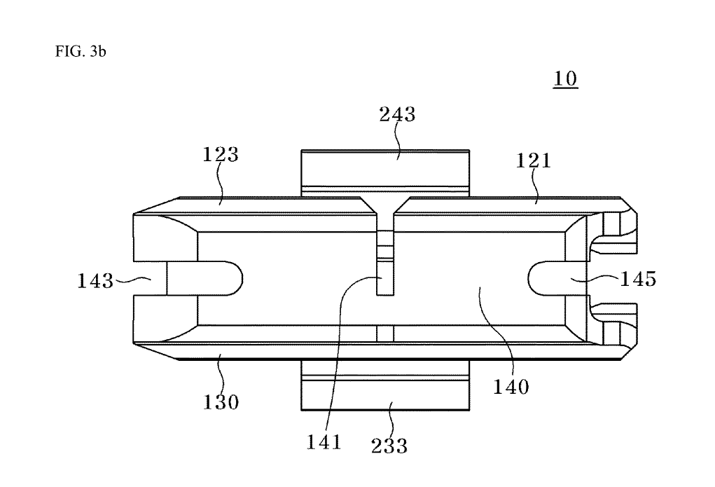

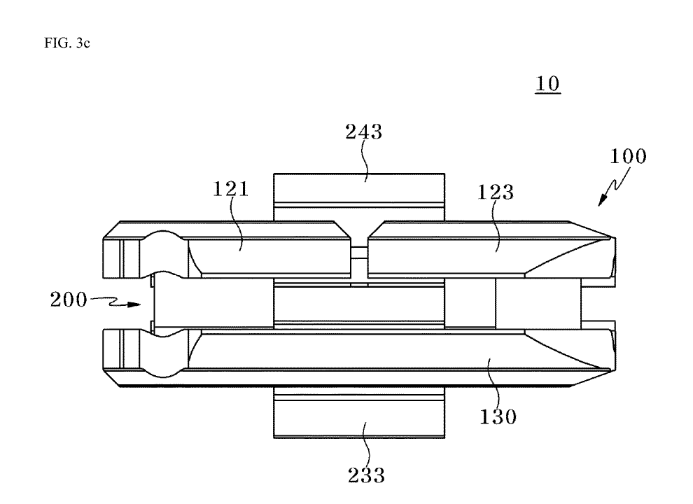

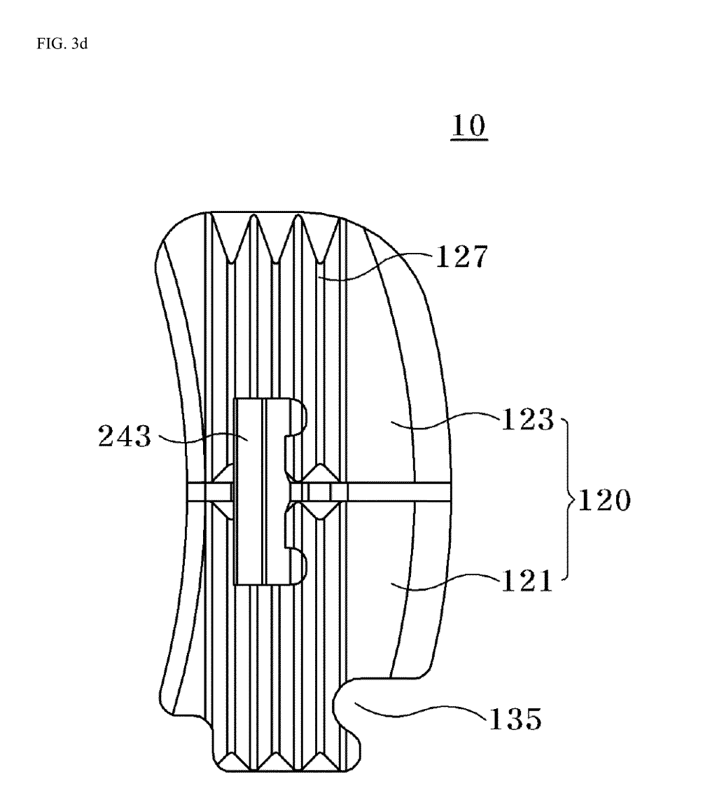

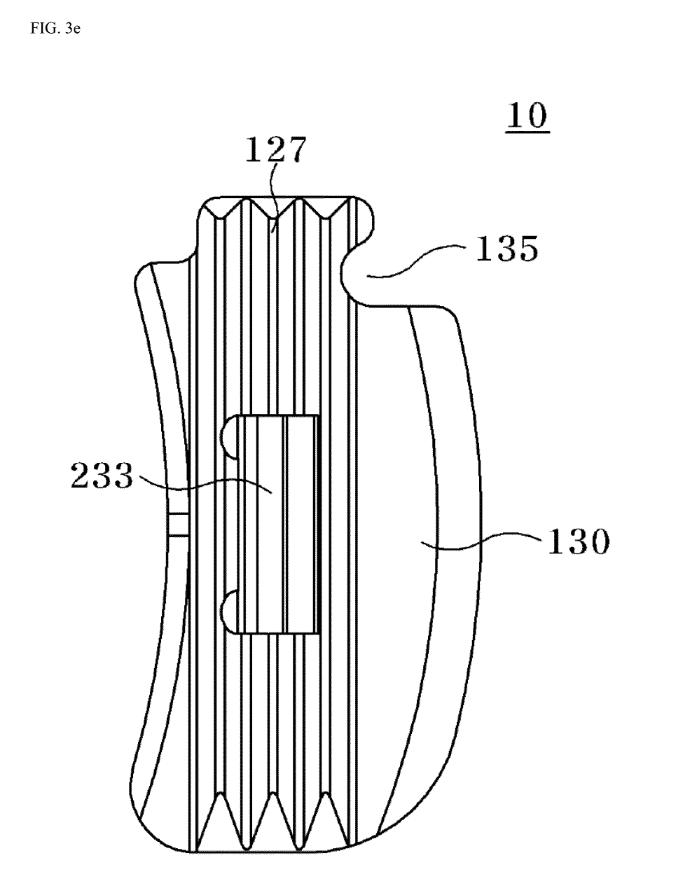

FIG. 3a is a left lateral view of a percutaneous intervertebral artificial disc replacement for minimal invasive surgery whose bumps are prominent through the top and bottom. FIG. 3b is a posterior view of a percutaneous intervertebral artificial disc replacement for minimal invasive surgery. FIG. 3c is an anterior view of a percutaneous intervertebral artificial disc replacement for minimal invasive surgery. FIG. 3d is a superior view of a percutaneous intervertebral artificial disc replacement for minimal invasive surgery. FIG. 3e is an inferior view of a percutaneous intervertebral artificial disc replacement for minimal invasive surgery.

Referring now to FIG. 3a, the artificial disc holding means 200 combined to the inner portion 131 of the artificial disc body 100 is rotated 90 degrees to the left by the inserting device 20. By rotating 90 degrees to the left side, the coupling blazes 243, 233 become more prominent through the opening 125 performed at the upper plate 120 and the lower plate 130. The artificial disc replacement 10 is sustained safely and isn't dislocated between adjacent vertebrae by engagement of the prominent coupling blazes due to rotating 90 degrees of the artificial disc holding means and the upper and lower vertebrae.

Turning to FIG. 3d, the coupling blaze 243 protrudes from the opening 125 performed at the first upper plate 121 and the second upper plate 123. FIG. 3e shows the coupling blaze 233 protrudes from the opening 125 performed at the lower plate 130.

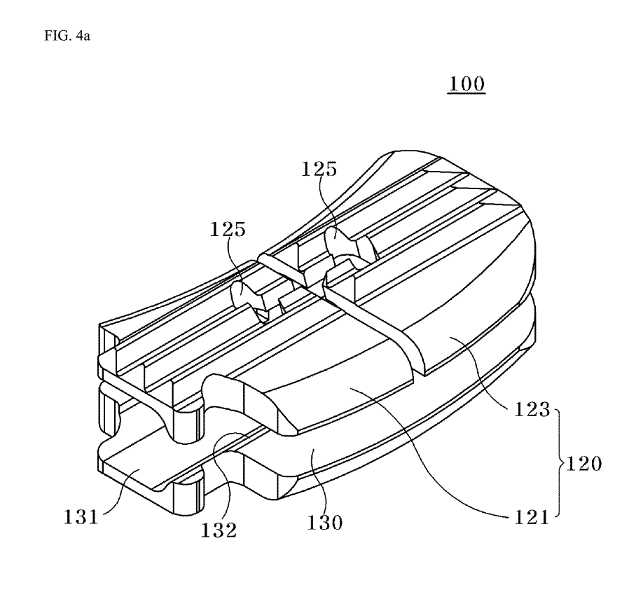

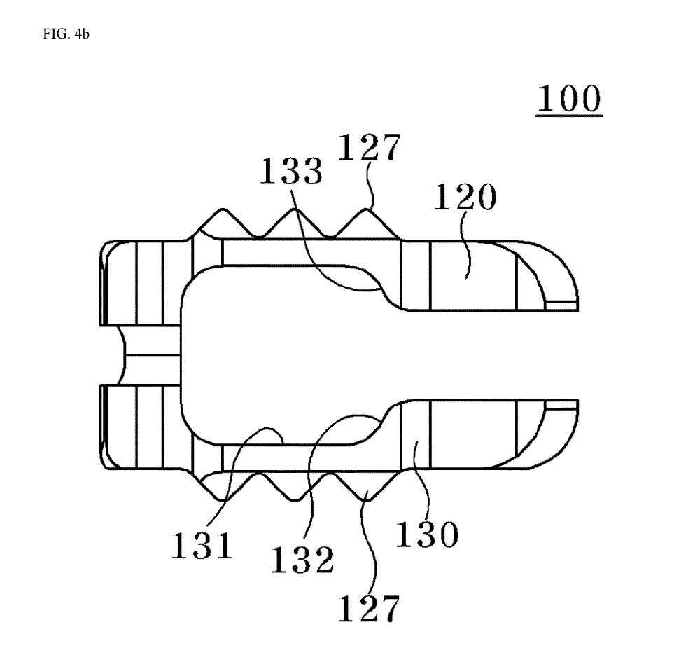

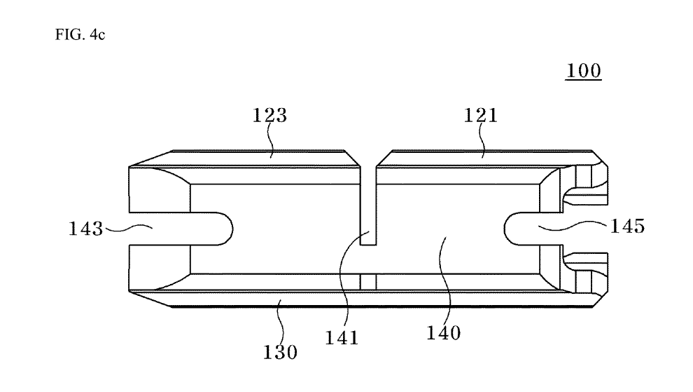

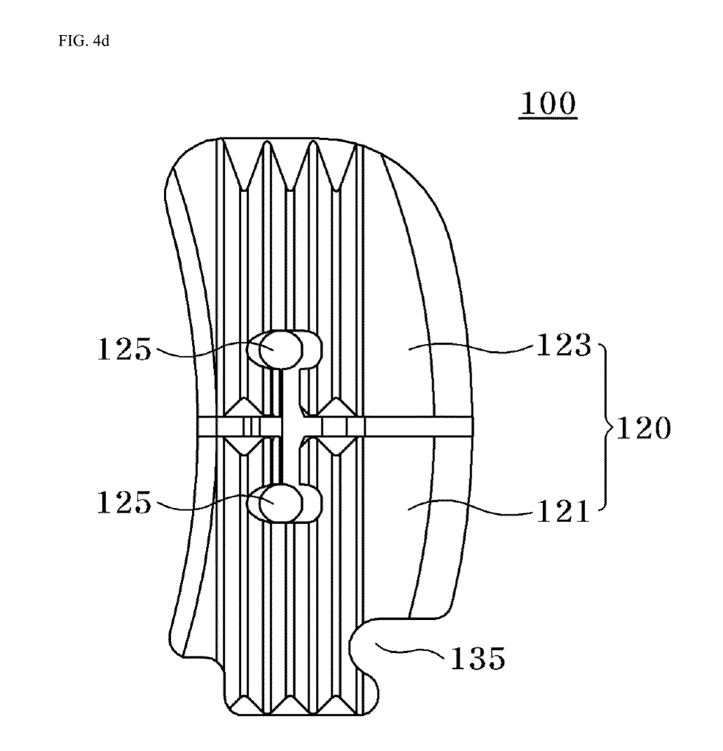

FIG. 4a is an isomeric view of an artificial disc body, FIG. 4b is a left lateral view of an artificial disc body, FIG. 4c is a posterior view of an artificial disc body, and FIG. 4d is a superior view of an artificial disc body, respectively.

Referring now to FIG. 4a, the opening 125 is performed at the upper plate 120 and the opening with same size performed at the lower plate 130. Specifically, the coupling blaze 243 composing the upper bump 240 protrudes through the opening 125 performed at the upper plate 120 and the coupling blaze 233 composing the lower bump 230 protrudes through the opening 125 performed at the lower plate 130.

Turning to FIG. 4b, an accepting portion 131 which accommodates the artificial disc holding means 200 is formed in the direction of the artificial disc body length inside of the artificial disc body 100. Bumps 132, 133 are formed to prevent separation of the artificial disc holding means accommodated. The artificial disc holding means can be detachably coupled to the accepting portion when broaden the gap between the upper plate and lower plate. The artificial disc holding means is not dislocated easily by the bumps 132, 133.

The artificial disc body 100 consists of the first upper plate 121, the second upper plate 123, the lower plate 130 and elastic intermediate connector 140. The upper plate 120 consists of the first upper plate 121 and the second upper plate 123 which is located separately on the left and right. Each openings 125 are performed on the first upper plate 121 and the second upper plate 123 and the two openings are connected and perform one opening. Many saw tooth shaped holding bumps 127 are performed on the first upper plate 121 and the second upper plate 123 and engaged with upper vertebrae.

The lower plate 130 also has opening 125 and many saw tooth shaped holding bumps which engage with lower vertebrae. The elastic intermediate connector 140 which connects the first and second upper plate 121, 123 to the lower plate 130 provides elastic force in an opposite direction of load in case the front portion of the first and second upper plate 121, 123 come down due to load of upper vertebrae.

Referring to FIG. 4c, the elastic intermediate connector 140 is a portion connecting the upper plate 120 and the lower plate 130 at the rear of the U-shaped artificial disc body. The elastic intermediate connector 140 has a certain length groove 141 in its middle side thereby the elastic intermediate connector 140 connected to the first upper plate 121 can be bent by a load on the first upper plate and the elastic intermediate connector 140 connected to the second upper plate 123 can be bent by a load on the second upper plate. The first or second upper plate 121, 123 can be separately bent down depends on loading locations; thereby the elastic intermediate connector 140 also can be bent separately based on the groove 141.

Furthermore, the elastic intermediate connector 140 has certain length grooves 145, 143 on the left and right side thereby the first upper plate 121 or the second upper plate 123 can be easily bent by weight force which loaded to left or right side of top of artificial disc body. In case of loading on the first upper plate, the first upper plate 121 can be bent to not only below but also left direction (direction of the groove 145). In case of loading on the second upper plate, the second upper plate 123 can be bent to not only below but also right direction (direction of the groove 143). The artificial disc replacement can have more natural mobility and can be more similar to a human disc according to the above structure.

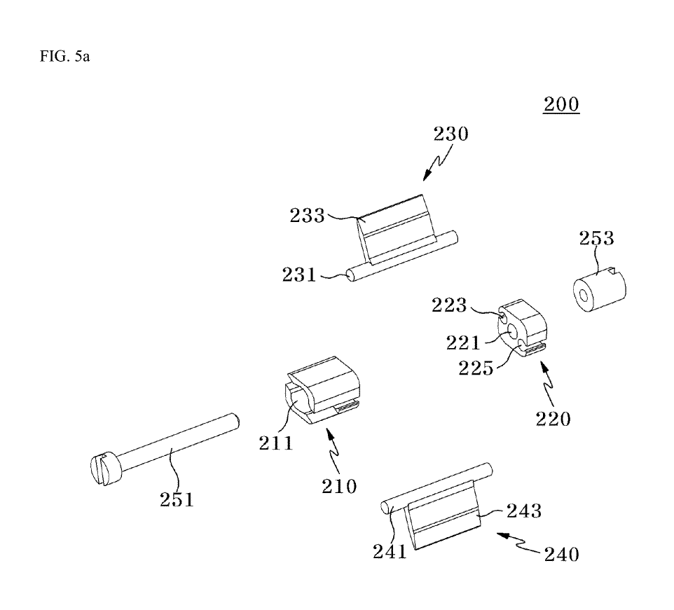

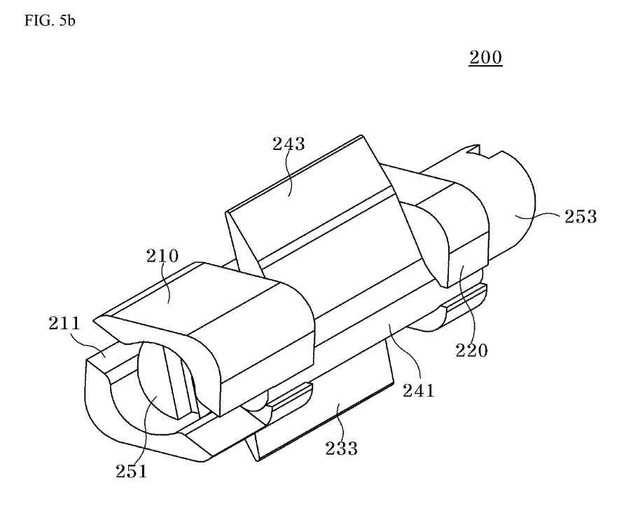

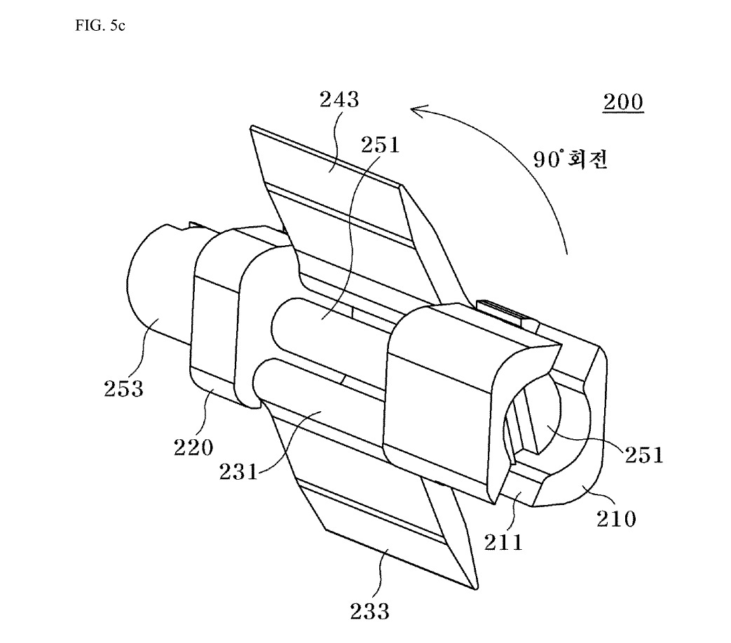

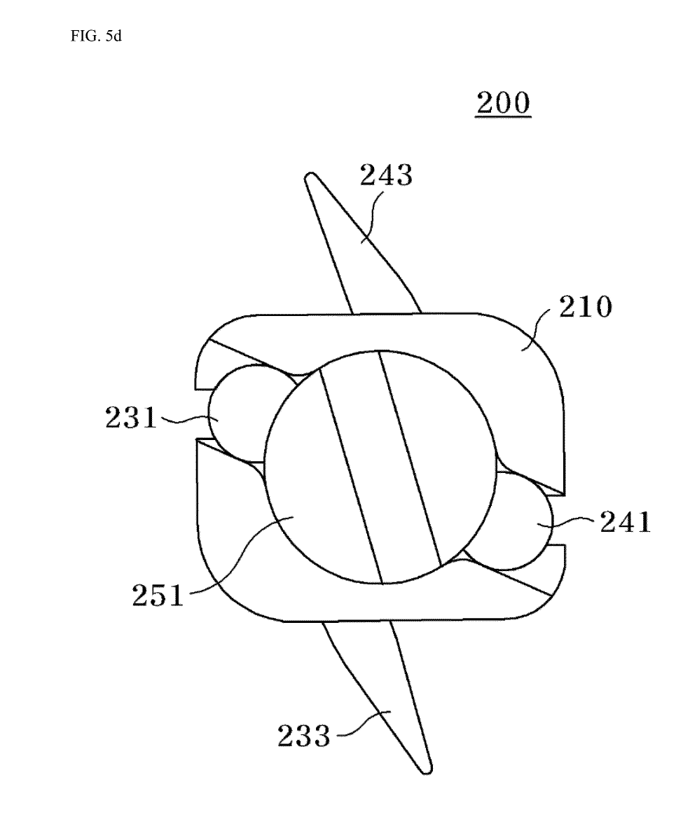

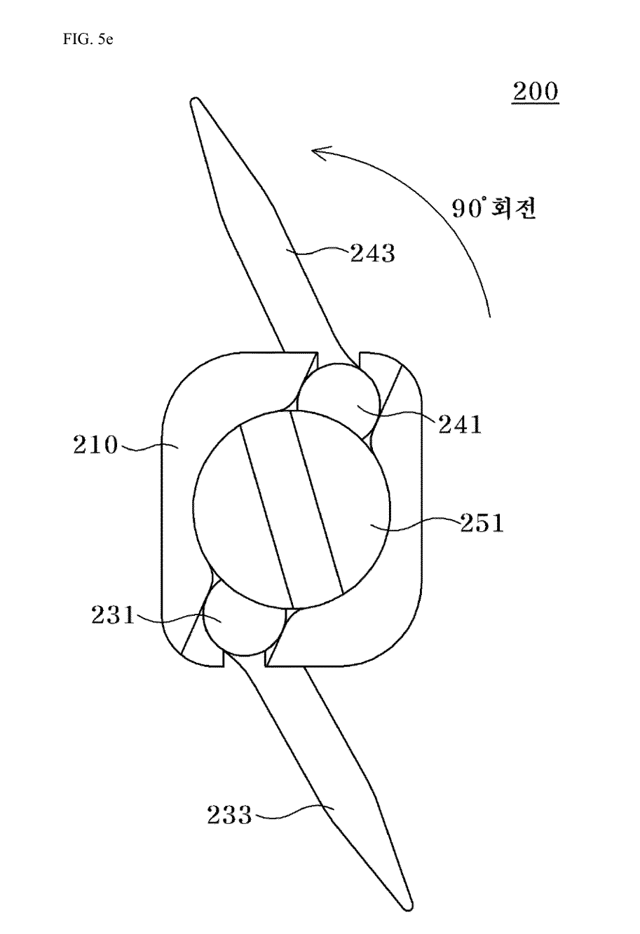

Next, an artificial disc holder or holding means is detailed. FIG. 5a is an exploded isomeric view of the artificial disc holding means; FIG. 5b is an isomeric view of the artificial disc holding means; FIG. 5c is an isomeric view of the artificial disc holding means which rotated by 90°, there the bump is prominent; FIG. 5d is a left lateral view of the artificial disc holding means; and FIG. 5e is a left lateral view of the artificial disc holding means which rotated by 90°, there the bump is prominent through an opening.

Turning now to FIG. 5a, the artificial disc holding means includes left body coupling portion, right body coupling portion, upper bump, lower bump, bolt portion, and nut portion. The upper bump includes a rod portion 241 and a coupling blaze 243. The lower bump includes a rod portion 231 and a coupling blaze 233.

The left body coupling portion 210 is coupled with left inside of the artificial disc body 100. A bolt through hole 221 has performed in the horizontal direction in the middle of the left coupling portion. There are each coupling hole 223, 225 at left and right side of bolt through hole 221.

The right body coupling portion 220 is coupled with right inside of the artificial disc body 100.

Bolt through hole 221 has performed in the horizontal direction in the middle of the right coupling portion. There are each coupling holes 223, 225 at left and right side of bolt through holes 221 facing opposite to coupling holes of the left coupling portion 210.

The upper bump 240 is located between left body coupling portion 210 and right body coupling portion 220. It rotatably coupled with right coupling hole 225 of left body coupling portion 210 and right body coupling portion 220. A coupling blaze 243 engages with upper vertebrae by being prominent through the opening 125 performed at upper portion of artificial disc body 100.

Concretely, the upper bump 240 is consisted of a rod portion 241 and a coupling blaze 243. Both end of the rod portion 241 are rotatably engaged with the coupling holes 225 of the left body coupling portion 210 and the right body coupling portion 220. The coupling blaze 243 is prominent through an opening 125 performed at the top of artificial disc, when the left and right body coupling portions 210, 220 were rotated by certain angle.

The lower bump 230 is located between left body coupling portion 210 and right body coupling portion 22. It rotatably coupled with left coupling hole 223 of left body coupling portion 210 and right body coupling portion 220. A coupling blaze 233 engages with lower vertebrae by being prominent through the opening 125 performed at lower portion of artificial disc body 100.

Concretely, the lower bump 230 is consisted of a rod portion 231 and a coupling blaze 233. Both end of the rod portion 231 are rotatably engaged with the coupling holes 223 of the left body coupling portion 210 and the right body coupling portion 220.

The coupling blaze 233 is prominent through an opening 125 performed at the bottom of artificial disc, when the left and right body coupling portions 210, 220 were rotated by certain angle.

The bolt portion 251 combines the left body coupling portion 210 and the right body coupling portion 220 by piercing bolt through hole 221 of the left body coupling portion 210 and the right body coupling portion 220 on condition that the upper and lower bumps 240, 230 are coupled with the left body coupling portion 210 on left and the right body coupling portion 220 on right of them.

The nut portion 253 is coupled with a part of the bolt portion 251 which is exposed to outside by piercing the right body coupling portion 220.

FIGS. 5b and 5d illustrate an artificial disc holding means combined to the inside of artificial disc body before the blazes 243, 233 are projected through the opening 125.

FIGS. 5c and 5e illustrate the artificial disc holding means 200 combined to the artificial disc body 100 is rotated 90 degrees to the left by the inserting device 20. By rotating 90 degrees, the coupling blaze 243 protrudes up through the opening 125 performed at the upper plate 120 and the coupling blaze 233 protrudes down through the opening 125 performed at the lower plate 130.

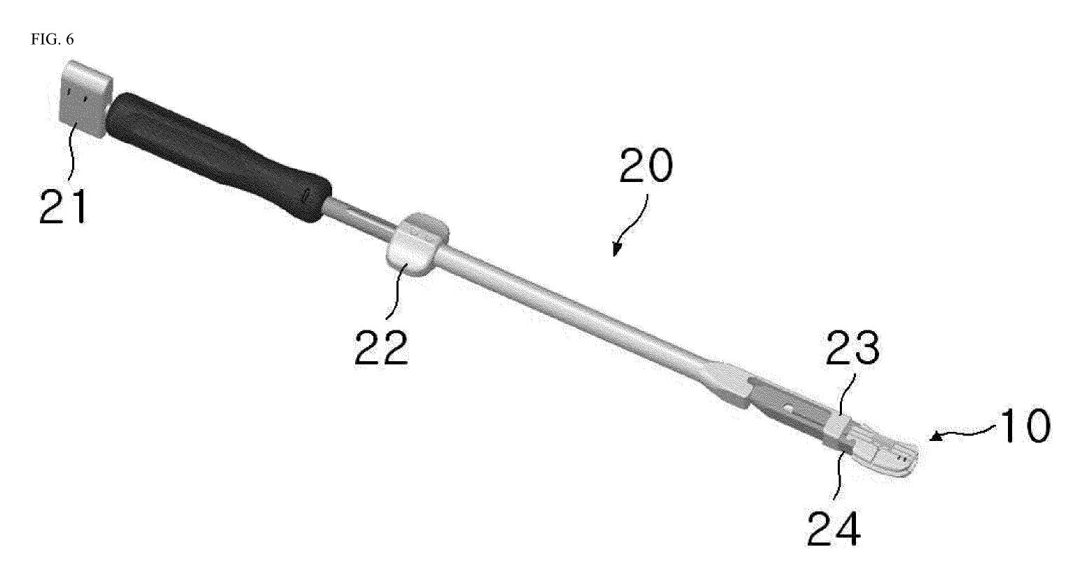

FIGS. 4d, 5a, 5b, 6 and 7 illustrate an inserting device which inserts the percutaneous intervertebral artificial disc replacement for minimal invasive surgery diagonally between adjacent vertebrae from back.

A concave shaped coupling groove coupling with the inserting device 20 is performed at the left and front side of the upper plate 120 of the artificial disc body 100 and the lower plate 130, and a groove 211 is performed at the side of left body coupling portion 210.

A coupling groove is facing opposite to a locking jaw 24 of the inserting device 20. The groove 211 is facing opposite to the coupling portion 25 of the inserting device 20 to rotate the artificial disc holding means 200 when the coupling portion 25 of the inserting device 20 rotate certain angle after coupling with the inserting device 20 for inserting the artificial disc replacement 10 between adjacent vertebrae.

A fastening portion 23 performed at one end move to the forward and backward by moving forward and backward of a moving portion 22 performed at the center of the inserting device 20.

The artificial disc replacement 10 combined with the inserting device 20 by the following procedure.

First, the fastening portion 23 moves backward. Secondly, the coupling portion 25 is fasten to the groove 211 of the left body coupling portion 210 of the artificial disc replacement 10. Thirdly, the fastening portion 23 moves toward the end of the procedure, the locking jaw 24 couple with the coupling groove 135 strongly.

The artificial disc replacement 10 coupled with the inserting device 20 is inserted diagonally between adjacent vertebrae from back by minimal invasive surgery. The artificial disc holding means engaged with the coupling portion 25 is rotated 90 degrees by rotation of the coupling portion 25 caused by rotation of the rotating portion 21 of the inserting device 20 after inserting artificial disc replacement between adjacent vertebrae.

By rotating 90 degrees to the left side, the coupling blazes 243, 233 protrude through the opening 125 performed at the upper plate 120 and the lower plate 130 and engage with upper and lower vertebrae thereby transplantation of the artificial disc replacement 10 between adjacent vertebrae is finished.

The inserting device should be disconnected from the artificial disc replacement 10 after transplant the artificial disc replacement 10. The fastening portion 23 will move to backward by moving the moving portion 22 to backward thereby the locking jaw 24 is disconnected from the coupling groove 135. And then the inserting device 20 can be removed.

For convenience, a list of the reference numbers is as follows:
10: A percutaneous intervertebral artificial disc replacement
20: An inserting device
21: A rotating portion
22: A moving portion
23: A fastening portion
24: A locking jaw
25: A coupling portion
100: An artificial disc body
120: An upper plate
121: The first upper plate
123: The second upper plate
125: An Opening
127: A holding bump
130: A lower plate
131: An accepting portion
133: A bump
135: A coupling groove
140: An elastic intermediate connector
141, 143, 145, 211: A groove
200: An artificial disc holding mean
210: A left body coupling portion
220: A right body coupling portion
221: A through hole
223, 225: Coupling holes
230: A lower bump
231, 241: Rod portions
233, 243: Coupling blazes
240: A nut portion

Wear and tear problem of the lower and upper plate of the artificial disc caused by the frequent friction can be handled by supporting vertebrae with elastic force. A protruded holding means engage with upper and lower vertebrae through openings at the top and bottom of artificial disc body thereby it prevents dislocation of artificial disc from adjacent vertebrae.

Furthermore, there are effects that minimize incision and bleeding by minimal invasive inserting the artificial disc with artificial disc replacement inserting device through patient's back.

Additionally, U-shaped artificial disc body has an effect of preventing dislocation of artificial disc replacement from adjacent vertebrae.

The various features and embodiments of the invention described herein may be used interchangeably with other feature and embodiments. Finally, while it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments which come within the spirit and scope of the present invention.

What is claimed is:

1. A percutaneous intervertebral artificial disc replacement for minimal invasive surgery comprising:
an artificial disc body having a top and bottom supporting an upper and a lower vertebrae, wherein the artificial disc body provides elasticity when inserted between adjacent vertebrae; and
an artificial disc holding means positioned inside the artificial disc body with parts engaging the upper and lower vertebrae through openings at the top and bottom of artificial disc body to prevent dislocation of the artificial disc from adjacent vertebrae, wherein the artificial disc holding means has two bumps each having a coupling blaze to contact the upper or lower vertebrae, wherein each coupling blaze is rotatable in the artificial disc holding means.

2. The artificial disc replacement of claim 1, comprising an accepting portion to accommodate the holding means of the artificial disc, wherein the accepting portion is formed in a direction of the artificial disc body length inside of the artificial disc body and wherein the bumps are formed to prevent separation of the artificial disc holding means.

3. The artificial disc replacement of claim 1, wherein the artificial disc holding means is detachably coupled to the accepting portion.

4. The artificial disc replacement of claim 1, wherein the artificial disc body comprises:
an upper plate including two first and second upper plates and having an opening and engaging the upper vertebrae;
a lower plate having an opening and engaging the lower vertebrae;
an elastic intermediate connector that connects the first and second upper plate to the lower plate and provides elastic force in an opposite direction of a load when the front portion of the first and second upper plates is compressed due to a load on the upper vertebrae.

5. The artificial disc replacement of claim 4, wherein the elastic intermediate connector comprises a vertical groove in a middle side to be bent separately by a force on each upper plate.

6. The artificial disc replacement of claim 5, wherein the above elastic intermediate connector comprises horizontal grooves that are bent by the force on either the left or right side of the top of the artificial disc body.

7. The artificial disc replacement of claim 1, wherein the artificial disc holding means comprising:
a left body coupling portion attached to a left inside of the artificial disc body;
a bolt-through hole in a horizontal direction in the middle of the left coupling portion with a coupling hole on each of the left and right side of bolt-through hole;
a right coupling portion coupled with right inside of the above artificial disc body;
a bolt through hole in the horizontal direction in the middle of the right coupling portion and coupling holes on the left and right side of the bolt-through holes facing opposite to coupling holes of the left coupling portion;
an upper bump located between the left body and right body coupling portions and rotatably coupled with right coupling hole of left and right body coupling portions;
a coupling blaze engaging the upper vertebrae by projecting through the opening performed at upper portion of artificial disc body;
a lower bump is located between the left and right body coupling portions and rotatably coupled with left coupling hole of left and right body coupling portions;
a coupling blaze engaging with lower vertebrae by projecting through the opening performed at lower portion of artificial disc body;
a bolt portion combines the left and the right body coupling portions by piercing the bolt through hole of the left and right body coupling portions when the upper and lower bumps are coupled to the left body coupling portion and the right body coupling portion; and
a nut portion is coupled with a part of the bolt portion which is exposed to outside by piercing the right body coupling portion.

8. The artificial disc replacement of claim 7, wherein the upper bump comprises:
a rotatable rod portion whose ends engage the coupling holes of the left body coupling portion and the right body coupling portion; and
another rod portion attached to the middle of the rotatable rod portion and including a coupling blaze which is prominent through an opening performed at the top of the artificial disc when the left and right body coupling portions are rotated by a predetermined angle.

9. The artificial disc replacement of claim 8, wherein the lower bump comprises:
a rotatable rod portion whose ends are engaged with the coupling holes of the left body coupling portion and the right body coupling portion; and
a coupling blaze which is prominent through an opening at the bottom of artificial disc when the left and right body coupling portions are rotated by a predetermined angle.

10. The artificial disc replacement of claim 8, comprising a groove matching a coupling portion of an inserting device when applied on the side of left body coupling portion to rotate the holding means of artificial disc by rotating the coupling portion of the inserting device when the artificial disc engaged with the inserting device.

11. A percutaneous intervertebral artificial disc replacement system for minimal invasive surgery comprising:
an artificial disc replacement inserting device; and
an artificial disc body to be positioned by the inserting device between an upper and a lower vertebrae, including:
openings at a top and a bottom of the artificial disc body, the artificial disc body supporting the upper and a lower vertebrae;
an artificial disc holder positioned inside the artificial disc body and engaging the upper and lower vertebrae through said openings to prevent dislocation of the artificial disc from adjacent vertebrae; and
two bumps each having a coupling blaze facing the upper or lower vertebrae,
wherein the artificial disc holder is insertable inside of the artificial disc body and each coupling blaze is engaged by rotating the artificial disc holder.

12. The system of claim 11, comprising an accepting portion formed in a direction of the artificial disc body length inside of the artificial disc body to accommodate the disc holder, wherein the bumps prevent separation of the artificial disc holder and wherein the artificial disc holder is detachably coupled to the accepting portion.

13. The system of claim 11, wherein the artificial disc body comprises:
an upper plate including two first and second upper plates and having an opening and engaging the upper vertebrae;
a lower plate having an opening and engaging the lower vertebrae;
an elastic intermediate connector connecting the first and second upper plate to the lower plate and providing elastic force in an opposite direction of a load when the front portion of the first and second upper plates experiences a load on the upper vertebrae.

14. The system of claim 13, wherein the elastic intermediate connector comprises a vertical or a horizontal groove adapted to be bent by a force on the artificial disc body.

15. The system of claim 11, wherein the above artificial disc holder comprises:
a left body coupling portion attached to a left inside of the artificial disc body;
a bolt-through hole in a horizontal direction in the middle of the left coupling portion with a coupling hole on each of the left and right side of the hole;
a right coupling portion coupled to the right inside of the artificial disc body;
a bolt-through hole in a horizontal direction in the middle of the right coupling portion and coupling holes on each of the left and right side of the bolt-through holes facing opposite to coupling holes of the left coupling portion;
an upper bump rotatably coupled to the left body and the right body coupling portion;
a first coupling blaze engaging with the upper vertebrae through an opening on the upper portion of artificial disc body;
a lower bump rotatably coupled to the left body and the right body coupling portion;
a second coupling blaze engaging with the lower vertebrae through an opening on the lower portion of artificial disc body;
a bolt portion combining the left and right body coupling portions by inserting the bolt through hole of the left body coupling portion and the right body coupling portion when the upper and lower bumps are coupled to the left and right body coupling portions; and
a nut portion coupled to a part of the bolt portion exposed to the outside by piercing the right body coupling portion.

16. The system of claim 15, wherein the upper bump comprises:
a rotatable rod portion whose ends engage the coupling holes of the left body coupling portion and the right body coupling portion; and
another rod portion attached to the middle of the rotatable rod portion and including a coupling blaze which is prominent through an opening performed at the top of the artificial disc when the left and right body coupling portions are rotated by a predetermined angle.

17. The system of claim 15, wherein the lower bump comprises:
a rotatable rod portion whose ends are engaged with the coupling holes of the left and right body coupling portions; and
a coupling blaze which is prominent through an opening at the bottom of the artificial disc when the left and right body coupling portions are rotated by a predetermined angle.

18. The system of claim 16, comprising a groove matching a coupling portion of an inserting device when applied on the side of left body coupling portion to rotate the holder by rotating the coupling portion of the inserting device when the artificial disc engaged with the inserting device.

19. An artificial disc replacement to be inserted between adjacent vertebrae, comprising:
a U shaped disc body to support an upper and a lower vertebrae and to provide elasticity when inserted between adjacent vertebrae;
a disc holder located inside of the disc body having protrusions engaging with upper and lower vertebrae through openings at the top and bottom of disc body and to prevent dislocation of the disc from adjacent vertebrae, wherein the protrusions engage with an upper and a lower vertebrae through openings at the top and bottom of artificial disc body; and
two bumps each having a coupling blaze facing the upper or lower vertebrae,
wherein the disc holder is insertable inside of the disc body and wherein each coupling blaze is engaged by rotating the disc holder.

20. The artificial disc replacement of claim 19, comprising an artificial disc replacement inserting device to perform minimally invasive placement of the artificial disc through a patient's back with minimal incision and bleeding.
